(19) United States
(12) Patent Application Publication
(10) Pub. No.: US 2021/0402211 A1
(43) Pub. Date: Dec. 30, 2021

(54) LIGHT-HYDROGEN THERAPY INCLUDING LIGHTING MODULE, LIGHTING APPARATUS, AND LIGHTING METHOD FOR PARKINSON'S DISEASE PATIENTS

(71) Applicant: Hung-Yu Lin, New Taipei City (TW)

(72) Inventor: Hung-Yu Lin, New Taipei City (TW)

(21) Appl. No.: 17/351,375

(22) Filed: Jun. 18, 2021

Related U.S. Application Data
(60) Provisional application No. 63/043,194, filed on Jun. 24, 2020.

(51) Int. Cl. A61N 5/06; A61K 33/00; A61K 9/00

(52) U.S. Cl. CPC A61N 5/0618; A61N 5/0622; A61N 5/062; A61K 33/00; A61K 9/0095; A61N 2005/0659; A61N 2005/0663; A61N 2005/0652; A61N 2005/0629; A61N 2005/0632; A61N 2005/0643; A61K 9/0073

(57) ABSTRACT

Provided is a light-hydrogen therapy including a lighting module, a lighting apparatus and a lighting method. The process of operation is to continuously or discontinuously emit near infrared rays or visible red light to the lower back part of a human head and next to the human neck, while in combination with drinking hydrogen water with dissolved hydrogen of 0.1 ppm to 6 ppm in a container or inhaling a hydrogen gas with 0.1% to 3.8% hydrogen in a container, to improve the body functions of PD patients.

### TECHNICAL FIELD

The present disclosure relates to lighting mechanisms, and in particular, to a lighting module, a lighting apparatus, and a lighting method suitable for Parkinson's disease (PD) patients.

### BACKGROUND

Parkinson's disease (PD) is the second most common neuro-degenerative disease, which affects approximately 1% of people over 60 years old. For people with PD, progression of symptoms leads to the impairment of the quality of life and a large economic burden on the patients themselves, their families, and the whole society. Currently, PD medication is symptomatic and is mainly based on exogenous dopaminergic supplement.

For a PD patient, the present disclosure provides a new light-hydrogen therapy (also called NIR+H₂ therapy) as an addition to the patient's regular PD medication, to help alleviate PD patient's PD symptoms and improve UPDRS (Unified Parkinson's Disease Rating Scale) score. The light-hydrogen therapy involves using both light therapy and hydrogen therapy simultaneously. In the followings, the current light therapy and hydrogen therapy for PD will be described, and the innovations of the present disclosure will be explained.

Light therapy is a treatment in which the skin of a person is exposed to an artificial light source. Since 1960s, light therapy has been reported to improve wound healing and reduce pain, inflammation, and swelling. Researches have shown that red to infrared light (or photobiomodulation) is neuroprotective in patients with PD. In the market, there are light therapy products that can be applied to PD patients, such as VieLight that administers near-infrared light to the brain transcranially and intra-nasally. The light-hydrogen therapy in the present disclosure uses light therapy as a part of the therapy. However, in the present disclosure, the light is projected into the brain through the opening of the foramen magnum, which allows the light to better reach brain cells without being blocked by the skull, giving better results.

Molecular hydrogen (H₂) has been found to be a potent and possibly therapeutic antioxidant in both in vitro and in vivo studies. Several methods can be used to ingest hydrogen into the body, such as drinking hydrogen-dissolved water (hydrogen water), injecting hydrogen-dissolved saline, and inhaling hydrogen gas through a ventilator circuit, facemask, or nasal cannula. For PD patients, a previous clinical trial showed that a hydrogen therapy, drinking 1000 mL/d of hydrogen water, for 48 weeks could reduce PD symptoms and improve UPDRS score. However, our research reveal that a hydrogen therapy alone is not that effective (at least not in a short time). On the other hand, when combining light therapy and hydrogen therapy, the improvement becomes significant and sustainable. Note that, without hydrogen therapy, a light therapy for a PD patient could be effective for a short period of time (e.g., a couple weeks); however, the benefit is not long lasting. Thus, the present disclosure is a joint use of the light and hydrogen therapies to create a new light-hydrogen therapy that can effectively improve the quality of life for PD patients.

### SUMMARY

In order to solve the foregoing problems of the prior art, the present disclosure discloses a lighting module, which comprises: a light-emitting device providing light in a continuous or discontinuous emission mode, wherein the light is near infrared rays or visible red light; and a container containing a hydrogen-containing fluid to work together with the light-emitting device via an intake of the hydrogen-containing fluid.

In the foregoing lighting module, the light-emitting device provides the light of a single wavelength.

In the foregoing lighting module, the light-emitting device provides the light of different wavelengths.

In the foregoing lighting module, the light-emitting device comprises one or more light-emitting diodes (LED) generating the near infrared rays of a single wavelength within a range from 600 to 1400 nanometers.

In the foregoing lighting module, the hydrogen-containing fluid is hydrogen water or hydrogen gas, wherein the hydrogen water is regular water dissolved with 0.1 ppm to 6 ppm of hydrogen and the hydrogen gas is regular air mixed with 0.1% to 3.8% of hydrogen.

In the foregoing lighting module, the present disclosure further comprises a power source electrically connected to the light-emitting device, where the power source provides a function of grounding (under a common condition) and is electrically connected to an external surface of the container or a ground of the container.

The present disclosure further provides a lighting apparatus, which comprises: a carrier; and the foregoing lighting module integrated into the carrier.

In the foregoing lighting apparatus, the carrier exhibits a chair structure.

The present disclosure further provides a lighting method, which comprises: providing a light source emitting light in a continuous or discontinuous emission mode on a lower back part of a head of a human body and next to a back of a neck of the human body, wherein the light emitted by the light source is near infrared rays or visible red light; and inhaling or drinking a hydrogen-containing fluid.

In the foregoing lighting method, the present disclosure further comprises providing the foregoing lighting module, wherein the light-emitting device of the lighting module serves as the light source, and the container of the lighting module contains the hydrogen-containing fluid.

In the foregoing lighting method, the light emitted from the light source penetrates through a foramen magnum and/or surrounding holes of the foramen magnum, and then enters the human brain.

It can be seen from the above that the lighting module, the lighting apparatus and the lighting method of the present disclosure provide near infrared rays of a single wavelength or different wavelengths within a range from 600 to 1400 nanometers to illuminate the area at the lower back part of a PD patient's head and next to the PD patient's neck, and combine with the PD patient's intake of a hydrogen-containing fluid (such as drinking hydrogen water in a container, or inhaling a hydrogen gas in a container) to improve the body functions of the PD patient. Therefore, the lighting module, the lighting apparatus and the lighting method of the present disclosure can be used to conduct light-hydrogen therapy on PD patients in a simple manner, and are convenient for PD patients to conduct the therapy at home.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-1 is the schematic diagram of the use of the lighting module of the present disclosure.

FIG. 1A-2 is the schematic diagram of the time axis of the light provided by the light-emitting device of the present disclosure.

FIG. 1B-1 is the schematic diagram of the first embodiment of the light-emitting device from the lighting module of the present disclosure.

FIG. 1B-2 is the schematic diagram of the circuit configuration of FIG. 1B-1.

FIG. 1B-3 is the schematic diagram of the duty cycle of FIG. 1B-2.

FIGS. 1C, 1D, 1E and 1F are the schematic diagrams of different implementations for FIG. 1B-3.

FIG. 2A is the schematic diagram of the use of FIG. 1B-1.

FIG. 2B is the schematic diagram of the second embodiment of the light-emitting device from the lighting module of the present disclosure.

FIG. 2C is the schematic diagram of the use of FIG. 2B.

FIG. 3A is the schematic diagram of the lighting module of the present disclosure.

FIG. 3B is the schematic diagram of the use of FIG. 3A.

FIG. 4 is the schematic diagram of another embodiment of the lighting module of the present disclosure.

FIG. 5 is the three-dimensional schematic diagram of the lighting apparatus of the present disclosure.

FIG. 6 is the schematic diagram of the explanation related to near infrared rays and H₂ for the mechanism of the Parkinson's disease treatment by the lighting method of the present disclosure.

### DETAILED DESCRIPTION OF THE EMBODIMENTS

The following embodiments illustrate the implementation of the present disclosure. Those skilled in this art can easily understand the other advantages and functions of the present disclosure from the content of the disclosure.

It should be noted that the structure, proportion, size, etc., shown in the drawings attached to this disclosure are used to explain the content of the disclosure for those familiar with the art to understand and read, and are not intended to serve as the limiting factors to restrict the implementation of the present disclosure. Any modifications in the structure, changes in proportion, or adjustments in sizes without affecting the functions and goals to be achieved by the present disclosure should still fall within the scope of the present disclosure. The terms such as “inner,” “outer,” and “one” cited in this disclosure are used for ease of description, but are not used to limit the implementation scope of the present disclosure. Changes or adjustments in their relative relationships, without substantial changes to the technical content, should also be regarded as being included in the implementation scope of the present disclosure.

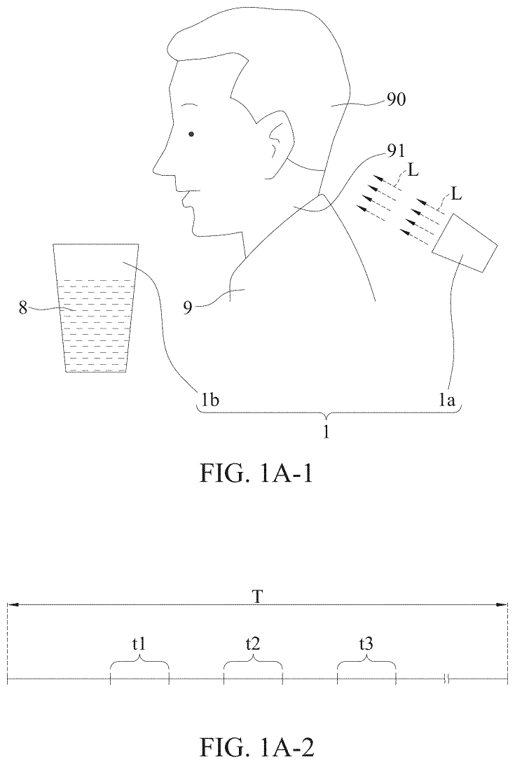

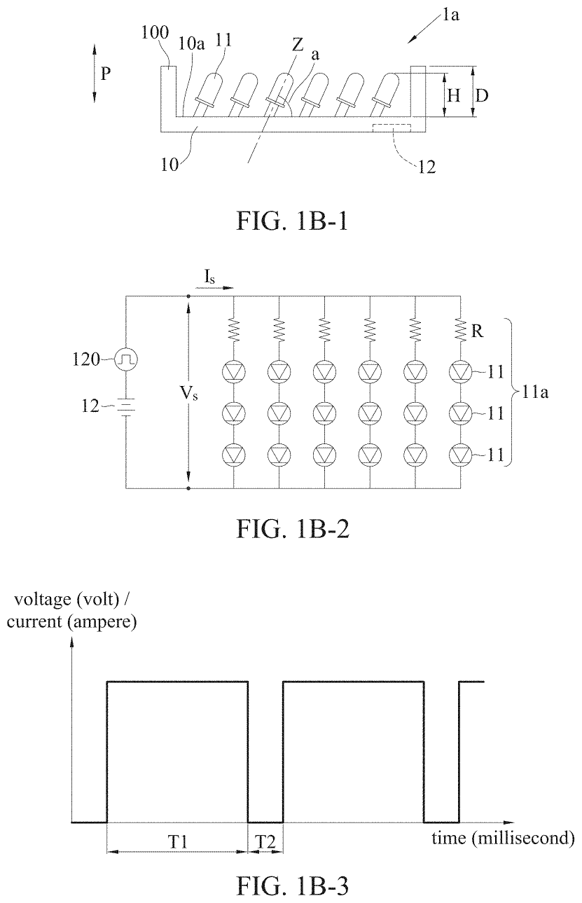

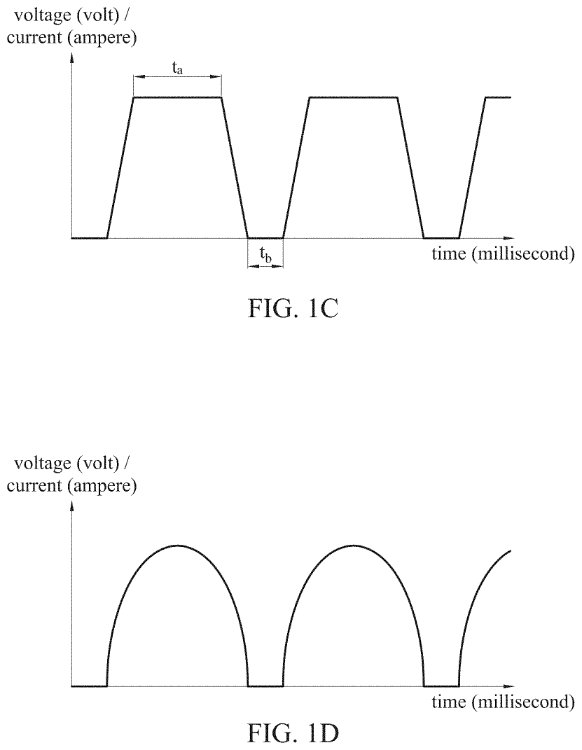

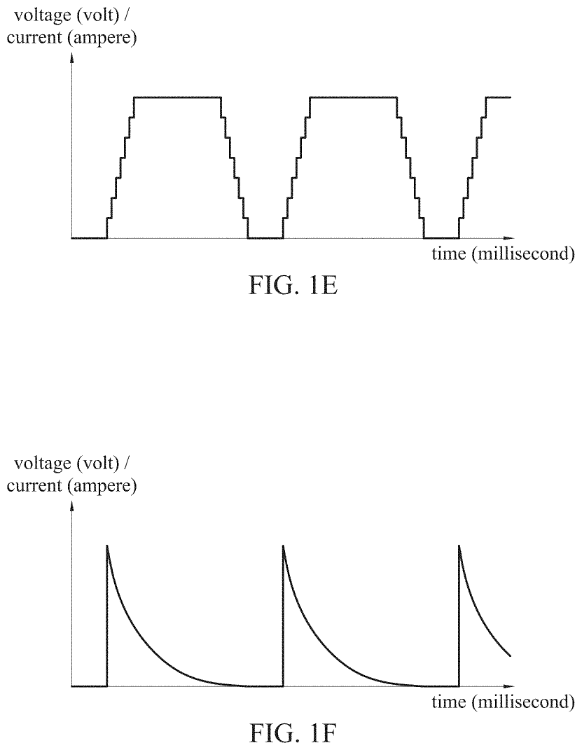

FIG. 1A-1 is the schematic diagram illustrating the lighting method featuring a lighting module 1 of the present disclosure applied to a human body. As shown in FIG. 1A-1, the lighting module 1 comprises a light-emitting device 1a and a container 1b.

The light-emitting device 1a provides light L in a continuous or discontinuous emission mode, wherein the light L is near infrared rays (NIR or simply near infrared) or visible red light. That is, the lighting intensity of the light-emitting device 1a is generated by various alternating current (AC) waveforms superimposed on a direct current (DC), and the levels of the AC and/or DC are adjustable. It should be noted that if the AC voltage is close to zero (≈0), or even equal to zero (=0), the current functions as a DC.

The container 1b is used to contain a hydrogen-containing fluid 8, 1200 cm³ (cc) of hydrogen water for example, to work together with the light-emitting device 1a via the intake of the hydrogen-containing fluid 8.

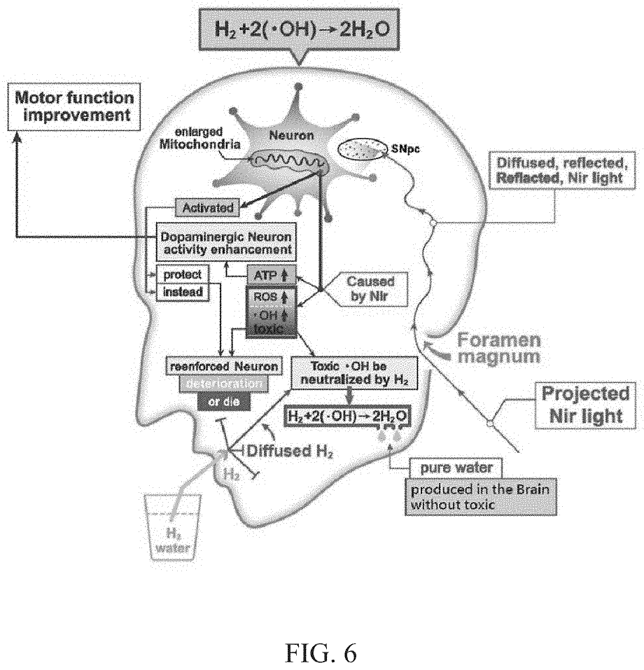

Therefore, when using the lighting module 1, a user 9 (such as a PD patient) uses the light-emitting device 1a as a light source, and allows the light L (such as near infrared rays or visible red light) to penetrate into the brain through the lower back part of a human head 90 and next (adjacent) to the human neck 91 in a continuous or discontinuous emission mode (that is, the light L penetrates through the foramen magnum). A part of the light L penetrates into the substantia nigra pars compacta (abbreviated as SNpc) in the midbrain. The photon energy of the light L is absorbed by the mitochondria inside the dopaminergic neurons to generate more adenosine triphosphate (ATP), thereby leading to the secretion of more dopamine from the dopaminergic neurons. Dopamine enhances exercise capacity, that is, physical activation (as shown in FIG. 6). ATP is the most important and direct energy source for cells, and ATP can enhance the motor function of PD patients. The user 9 also ingests the hydrogen-containing fluid 8 by drinking the hydrogen water (regular water with 0.1 ppm to 6 ppm of dissolved hydrogen) in the container 1b, or inhaling the hydrogen gas (regular air mixed with 0.1% to 3.8% hydrogen) in the container 1b. Note that, when the NIR light L acts on the mitochondria in the midbrain cells, the proliferated ATP is accompanied by the generation of a large amount of very toxic ⋅ OH free radicals during the oxidative metabolism, which could kill the nerve cells themselves. To avoid this situation, the user 9 ingests hydrogen molecules H2, which exhibits a strong power in neutralizing ⋅ OH in the brain to become non-toxic water. As such, the problem of degeneration or death of various cells subject to the high-intensity oxidation of ⋅ OH can be avoided. This can keep the benefits of using light therapy without having undesired bad results. Therefore, the curative effect of light therapy on PD patients can be greatly enhanced.

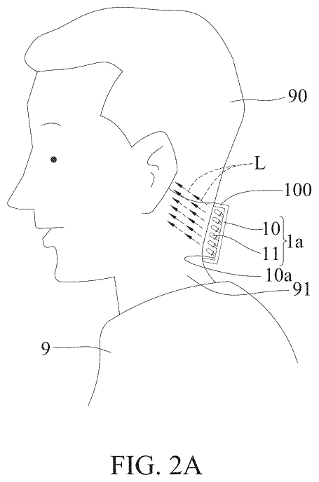

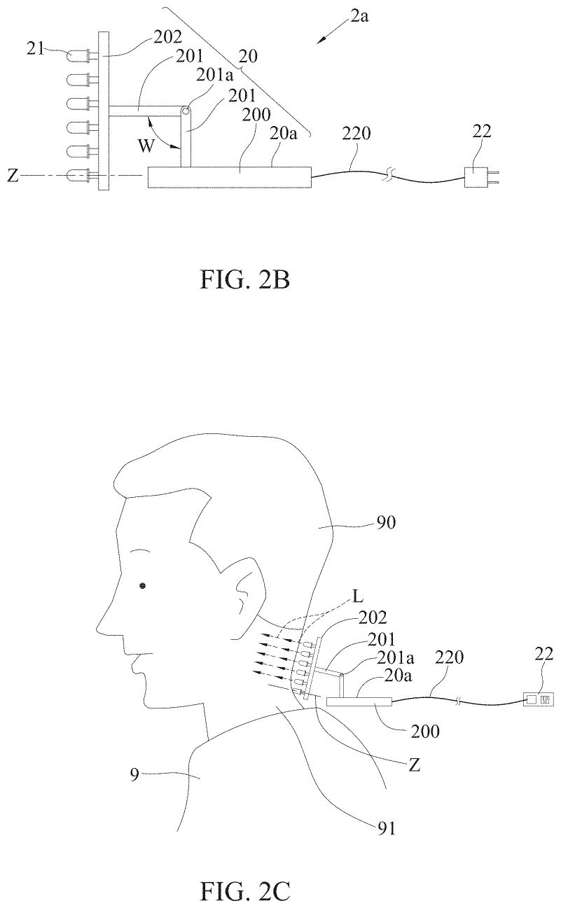

Referring to FIG. 1A-2, in an embodiment, the discontinuous light emission is to supply the light L intermittently during the entire period T of the lighting process. For example, in the overall period T of the lighting process, the supply of the light L is interrupted for at least one interval of t1, t2 and t3 (as shown in FIG. 1A-2). Specifically, the overall period T is 15 minutes, and the time interval for t1, t2 and t3 may be several seconds or several milliseconds (ms). It is understood that the light L would look blinking if the interruptions exist at multiple time intervals and last between tens of milliseconds and several seconds, for example.

Furthermore, a human brain is covered with a skull which blocks light. However, a foramen magnum exists near the area at the lower back part of a human head 90 and next to the neck 91, and is a large oval opening. Thus, the light L can penetrate through the foramen magnum and/or the surrounding holes thereof, and enters the brain. While the light L travels in a straight line in the air, the light L travels in many different directions inside the brain via reflection, refraction and diffraction. As such, photons can distribute themselves in the brain, especially can reach to the substantia nigra pars compacta (SNpc), that is, the dense area of the substantia nigra cells in the brain. When the SNpc is lighted by the photons of near infrared rays or visible red light, the amount of secreted dopamine would be increased.

In addition, the hydrogen-containing fluid 8 is hydrogen water (regular water dissolved with 0.1 ppm to 6 ppm of hydrogen) or hydrogen gas (regular air mixed with 0.1% to 3.8% of hydrogen). Normally, the water saturation of dissolved hydrogen is from 1.5 to 1.6 ppm at room temperature.

Moreover, hydrogen can scavenge reactive oxygen free radicals (reactive oxygen species, referred to as ROS) in the body, where the reactive oxygen species contain a large amount of highly toxic ⋅ OH free radicals. In short, the illumination of near infrared rays to cells in the brain would generate good results and bad results. The good result is the proliferation of ATP (which would activate brain cells and enhance physical strength), and the bad result is the generation of ⋅ OH free radicals. Therefore, hydrogen water is used to provide hydrogen molecules for a reductive reaction (i.e., H₂+2(⋅ OH)→2H₂O) to take place so as to reduce the ⋅ OH free radicals in the brain to become non-toxic water (H₂O). As such, the purpose of keeping the good result while eliminating the bad result is achieved (see FIG. 6 for details).

Therefore, the lighting module 1 of the present disclosure not only can stimulate the secretory neurons in the substantia nigra pars compacta (SNpc) in the brain to increase the secretion of dopamine, but can also eliminate the ⋅ OH free radicals generated in the lighting process through the ingestion of hydrogen. Therefore, the module can improve the symptoms of PD, restore the patient's motor functions, and allow the patient to walk normally again. Compared to the prior art, the lighting module 1 of the present disclosure is a simple device to conduct light-hydrogen therapy on a PD patient, such that the lighting module 1 of the present disclosure allows the patient to conduct the therapy at home. In other words, there is no need to go to specific places (such as clinics or hospitals) for PD related treatments.

FIGS. 1B-1 and 2A illustrate the first embodiment of the light-emitting device 1a as a light source from the lighting module 1 of the present disclosure.

As shown in FIGS. 1B-1 and 1B-2, the light-emitting device 1a comprises a carrier 10, at least one light-emitting component 11, and a power source 12.

The carrier 10 is of a flat body (e.g., plate body), such as a circuit board. The carrier 10 has a carrier surface 10a for the assembly of (or bonding) the light-emitting components 11.

In an embodiment, the edges of the carrier surface 10a of the carrier 10 are surrounded by flat vertical walls 100.

The assembled light-emitting components 11 on the carrier surface 10a are obliquely oriented with respect to the carrier surface 10a. The central axis Z of each light-emitting component 11, when in use, presents an included angle a of less than or equal to 90° with respect to the carrier surface 10a, such as 35° or 80°.

In an embodiment, the light-emitting components 11 are diodes, such as light-emitting diodes or laser diodes. The light emitted by the light-emitting components 11 is single-wavelength near infrared rays, of which the wavelength is between 600 nanometers (nm) and 1400 nm, and preferably between 700 nm and 1100 nm. For example, the light-emitting device 1a would become a near infrared projector if a plurality of the light-emitting components 11 are arranged in an array.

Furthermore, the projected height H of each light-emitting component 11 in the vertical direction (e.g., the direction of the arrows P in FIG. 1B-1) with respect to the carrier surface 10a is shorter than the height D of the walls 100 with respect to the carrier surface 10a.

The power source 12 is supplied for example by a battery, and is electrically connected to the light-emitting components 11. The current from the power source 12 is supplied intermittently to allow the light-emitting components 11 to provide near infrared rays in a continuous or discontinuous emission mode.

In an embodiment, the power source 12 is free from supplying a continuous direct current, but supplies an intermittent direct current to turn on and off the light-emitting components 11 intermittently. The light L generated by the intermittent direct current presents greater efficiency in the proliferation of ATP than the light generated by a continuous direct current. This not only can enhance the activation of mitochondria, but also further activates cells.

Furthermore, the circuit configuration of the light-emitting device 1a is to superimpose a pulse modulation 120 on a direct current (DC) power source (as shown in FIG. 1B-2) to generate the required duty cycle (the square wave shown in FIG. 1B-3). For example, in a single duty cycle shown in FIG. 1B-3, the current Is (FIG. 1B-2) in the on-state T1 is non-zero (i.e., the power source 12 is turned on) and the current Is in the off-state T2 is zero (i.e., the power source 12 is turned oft); the time interval of T1 is 160 milliseconds, and the time interval of T2 is 40 milliseconds. Thus, the duty cycle is 80%. Specifically, in the circuit configuration of the light-emitting device 1 a, the light-emitting components 11 are arranged in an array with the plurality of light-emitting components 11 in series connection with a resistor R to form a light-emitting set 11a, and a plurality of light-emitting sets 11a are connected in parallel with the power source 12 (e.g., the voltage source Vs and the current source Is shown in FIG. 1B-2).

It is understood that the duty cycle can also be implemented with different waveforms, such as the trapezoidal wave shown in FIG. 1C, the cosine-squared wave shown in FIG. 1D, the staircase wave shown in FIG. 1E, or other waveforms. Another spike waveform is shown in FIG. 1F (the waveform of the current from a discharged capacitor that was charged first). Among these, FIG. 1C would demonstrate a triangular-wave if the time intervals to and tb are both zero. The staircase wave shown in FIG. 1E can be generated via digital synthesis, that is, a staircase-like waveform generated by a D/A converter (Digital-to-Analog Converter). Alternatively, a pulse-width modulation (referred to as PWM) would also work.

In addition, the ground of the power source 12 and the external surface (such as the housing) of the container 1b or the ground of the container 1b are electrically connected.

As shown in FIG. 2A, a user 9 places the wall 100 of the carrier 10 against the area at the lower back part of the head 90 and next to the neck 91 to allow the light L from the light-emitting components 11 to enter the brain.

Therefore, the lighting module 1 of the present disclosure provides the light-emitting device 1a as a light source, and the light-emitting components 11 are oriented obliquely with respect to the carrier surface 10a. The carrier 10 is placed against the area at the lower back part of the head 90 and next to the neck 91. This would benefit the penetration of the light L from the light-emitting components 11 through the foramen magnum and/or the surrounding holes thereof and then into the mid-brain area (i.e., SNpc) of the brain, allowing more photons to stimulate the secretory neurons in the substantia nigra pars compacta to increase the secretion of dopamine

FIG. 2B is the schematic diagram of a second embodiment of the light-emitting device 2a as a light source in the lighting module of the present disclosure. The difference between the second embodiment and the previous embodiment lies in the connection relationship between a carrier 20 and light-emitting components 21. The other parts of the structure are basically the same, and will not be repeated here for brevity.

FIG. 2B shows the light-emitting components 21 arranged in a manner to allow the light-emitting components 21 to be separated from a carrier surface 20a.

In an embodiment, the carrier 20 is a supporting structure comprising a carrier base 200 with a carrier surface 20a, a rod set 201 with one end fixed on the carrier surface 20a, and a supporting base 202 connected to the other end of the rod set 201. Specifically, the rod set 201 comprising a rotating shaft 201a would allow an inclination angle of the supporting base 202 with respect to the carrier surface 20a. The inclination angle is adjustable and subject to change upon requirements (the rotation direction W is shown in FIG. 2B).

Furthermore, the light-emitting components 21 are vertically assembled on the supporting base 202, which would make the central axis Z of each light-emitting component 21, when in use, perpendicular to the supporting base 202.

In addition, the power source 22 is an environmental socket (a plug and a socket shown in FIGS. 2B and 2C, respectively), which delivers power to the light-emitting components 21 through at least one wire 220. It is understood that the power source 22 could also be a battery.

When in use, as shown in FIG. 2C, the user 9 adjusts the inclination angle of the supporting base 202 with respect to the carrier surface 20a through a shaft 201a of the rod set 201 to allow the central axis Z of each light-emitting component 21 to point to the area at the lower back part of the user's head 90 and next to the user's neck 91. Hence, the light L from the light-emitting components 21 can enter the brain.

Therefore, the light-emitting device 2a served as the light source of the lighting module 1 of the present disclosure allows the adjustment of the orientations of the light-emitting components 21 to be oblique with respect to the carrier surface 20a. Therefore, the light L generated by the light-emitting components 21 can pass through the foramen magnum and/or the surrounding holes thereof to illuminate the mid-brain, which would lead to the proliferation of ATP and allow the secretory neurons in the substantia nigra pars compacta of the mid-brain to increase the secretion of dopamine.

In addition, it is understood that the rod set 201 can be a deformable body, such as a flexible metal or plastic, to allow the rod set 201 to be bent arbitrarily. Alternatively, the rod set 201 can also be a positioning type, that is to fix the rod set 201 at a specific position or angle (which would allow the central axis Z of each light-emitting component 21 to point to the area at the lower back part the head 90 and next to the neck 91). The supporting base 202 would be fixed and oriented obliquely with respect to the carrier surface 20a (for example, the configuration shown in FIG. 2C), and the position of the supporting base 202 is free from being adjustable (for example, disallowing the configuration shown in FIG. 2B). As a result, the light-emitting components 21 are oriented obliquely with respect to the carrier surface 20a. Therefore, the concept of the embodiment of FIG. 2B is nearly the same as that of the first embodiment.

In other embodiments, the light-emitting devices 1a, 2a can also provide the light L of different wavelengths. For example, the light-emitting devices 1a, 2a could comprise three different types of light-emitting components 11, which emit near infrared rays of wavelengths 630 nm, 810 nm and 940 nm, respectively. Therefore, when in use, the three types of light-emitting components 11 simultaneously emit the light L of different wavelengths.

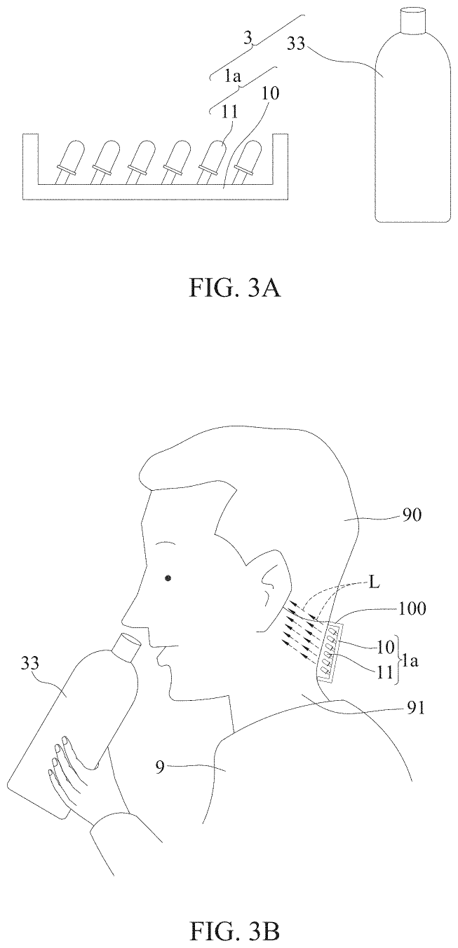

FIG. 3A is the schematic side view of the lighting module 3 of the present disclosure. The lighting module 3 of this embodiment uses the light-emitting device 1a from the first embodiment as a light source to work together with a container 33. The structure is the same and will not be repeated here for brevity.

The lighting module 3 comprises a light-emitting device 1a and a container 33 to contain a fluid.

In an embodiment, the container 33 can be, for example, a bag or a bottle, and is used to contain a liquid or a gas, such as hydrogen water or hydrogen gas. For example, the bottle-like container 33 can be a general container or an electric appliance capable of electrolyzing a solution.

When in use, as shown in FIG. 3B, the user 9 first uses the light-emitting components 11 to emit the light L in a continuous or discontinuous mode into the brain through the area at the lower back part of the head 90 and next to the neck 91 of the user 9, and then inhales the hydrogen gas in the container 33 (or drinks the hydrogen water in the container 33).

Therefore, with the combination of the light-emitting device 1a and the container 33, the lighting module 3 of the present disclosure can not only stimulate the secretory neurons in the substantia nigra pars compacta of the brain to increase the secretion of dopamine, but also eliminate the active oxygen free radicals produced during the process of stimulating the substantia nigra pars compacta in the midbrain by drinking the hydrogen water or inhaling the hydrogen gas. Therefore, compared to the prior art, the lighting module 3 of the present disclosure can be used to conduct light-hydrogen therapy for a PD patient with a simple appliance. Moreover, it is convenient for PD patients to conduct the therapy at home. That is, there is no need to go to specific places (such as clinics or hospitals) for PD related treatments.

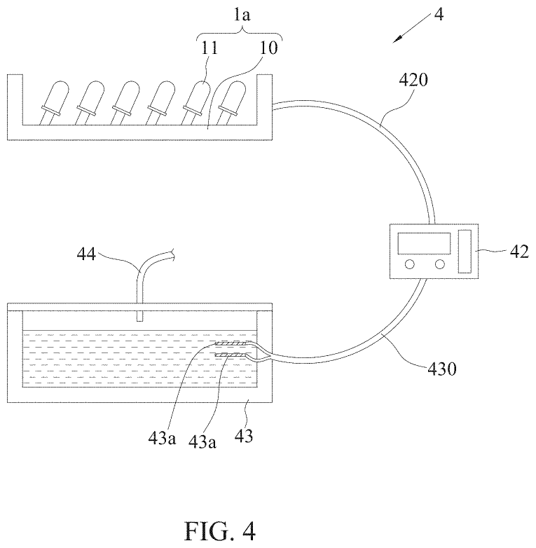

FIG. 4 is the schematic diagram of another embodiment of the lighting module 4 of the present disclosure. The difference between this embodiment and the foregoing embodiments lies in the design of the container 43. The other parts of the structure are basically the same, and will not be repeated here for brevity.

As shown in FIG. 4, the container 43 is an electrolytic tank, which provides a plurality of electrodes 43a, and is electrically connected to the power source 42 through at least one wire 430.

In an embodiment, the power source 42 is a generator or an indoor power socket. Power is delivered to the light-emitting components 11 through at least one wire 420.

Furthermore, the container 43 is an electrical appliance. Both the light-emitting device 1a and the container 43 are electrically connected (for example, grounded) to the power source 42. Hence, hazards such as the generation of electrostatic and subtle sparks which may lead to fire can be avoided. Therefore, the safety issue for the operation of the lighting module 4 of this embodiment is addressed.

When in use, the water in the container 43 is electrolyzed first by the power source 42 and turned into the required hydrogen water or hydrogen gas. The light from the light-emitting components 11 is emitted in a continuous or discontinuous mode into the brain through the area at the lower back part of the head and next to the neck. The user drinks the hydrogen water or inhales the hydrogen gas in the container 43 to eliminate the ⋅ OH free radicals inside the body.

Therefore, the lighting module 4 of the present disclosure employs the power source 42 to produce a hydrogen-containing fluid 8 shown in FIG. 1A-1. The power source 42 also provides the power for the light-emitting device 1a to generate light. This not only can stimulate the secretory neurons in the substantia nigra pars compacta inside the brain to increase the secretion of dopamine, but also can eliminate the active oxygen free radicals produced during the process of stimulating the substantia nigra pars compacta inside the brain. Therefore, compared to the prior art, the lighting module 4 of the present disclosure combines simple appliances to conduct light-hydrogen therapy. Therefore, it is convenient for PD patients to use the lighting module 4 at home. That is, the PD patients can receive the benefits of the light-hydrogen treatments provided by the lighting module 4 without having to go to specific places (such as clinics or hospitals) for related lighting mechanisms.

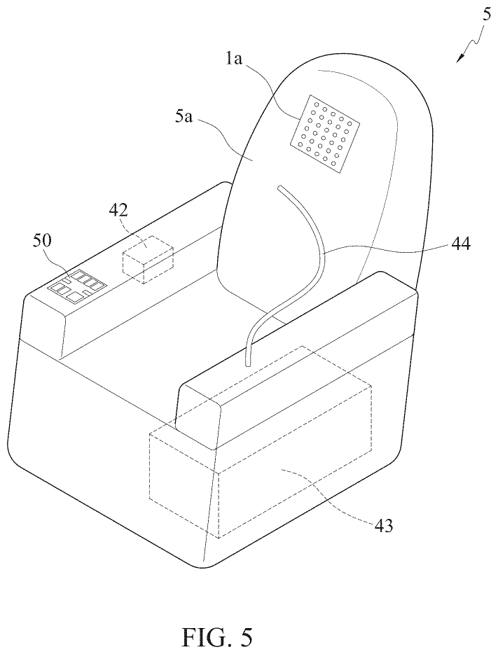

FIG. 5 is the three-dimensional schematic diagram of the lighting apparatus 5 of the present disclosure. As shown in FIG. 5, the lighting apparatus 5 is to integrate the lighting modules 1, 3, 4 into a carrier 5a.

In an embodiment, the carrier 5a is or exhibits a chair structure, in which the lighting module 4 shown in FIG. 4 is integrated. For example, the carrier 5a may be equipped with a control interface 50 for the user to control the lighting module 4 to function properly. Specifically, the light-emitting device 1a is placed near the upper end of the seat, the container 43 is placed underneath the seat, and the power source 42 is placed inside or outside the chair structure upon requirements (such as the location underneath the armrest area of the chair structure). It is understood that a suction tube 44 is subject to pulls upon requirements to facilitate the user to drink the hydrogen water or to inhale hydrogen gas.

When in use, the water in the container 43 is electrolyzed first by the power source 42 and turned into the required hydrogen water or hydrogen gas. The light from the light-emitting components 11 is emitted through the area at the lower back part of the head and next to the neck, and then into the brain. The user uses the suction tube 44 to drink the hydrogen water or inhale the hydrogen gas in the container 43.

Therefore, the lighting apparatus 5 of the present disclosure is designed for PD patients to operate the device at home through the design of the carrier 5a. Compared to the prior art, the lighting apparatus 5 of the present disclosure can serve the function of conducting light-hydrogen therapy for PD patients by using a simple appliance. Therefore, there is no need to go to a specific place for PD related treatments.

In other embodiments, the carrier 5a can also be equipped with the lighting module 3 shown in FIG. 3A or the other types of lighting modules. A fixture for the container 33 is mounted on top of the armrest area of the chair. The user uses the suction tube 44 to inhale the hydrogen gas in the container 33 (or to drink the hydrogen water in the container 33). It is understood that the structure of the carrier can be designed upon requirements, such as a frame structure, and is not restricted to a chair structure.

Therefore, the present disclosure provides light-hydrogen therapy for Parkinson's disease, which requires illumination on the head of a PD patient with near infrared rays of appropriate intensity, and drinks hydrogen water or inhales hydrogen gas. During the process of emitting light into the head, the current provided by the power source to produce near infrared rays can be a direct current or an intermittent current. The feature of the present disclosure is to provide PD patients with the illumination of near infrared rays and the intake of the hydrogen molecules at the same time or at different times. It should be noted that the stated “at different times” refers to the intake of the hydrogen molecules within 24 hours before or after the illumination of near infrared rays. However, the intake at the same time is preferred.

As shown in FIG. 6, mitochondria illuminated by near infrared rays would lead to the proliferation of ATP, which is accompanied by the generation of ⋅ OH hydroxyl radicals. ⋅ OH would induce cytolysis. Since ⋅ OH presents strong oxidation, a chemical reaction would occur as follows if hydrogen molecules are introduced (such as inhaling or drinking) at the same time:

H₂+2(⋅ OH)→2H₂O

In other words, when a non-toxic hydrogen molecule and two highly toxic hydroxyl radicals (⋅ OH hydroxyl radicals) are mixed in the brain, the hydrogen molecule and the hydroxyl radicals quickly react on each other to become non-toxic water inside the brain. It not only can avoid killing mitochondria, the illumination of near infrared rays would also strengthen mitochondria and their cells. As a result, more dopamine is secreted. In short, by keeping the good result (that is, the proliferation of dopamine by ATP) and eliminating the bad result (the neutralization of ⋅ OH to become pure water with no toxic ⋅ OH left in the brain), neurodegenerative diseases, such as Parkinson's disease, can be effectively treated.

In summary, the lighting apparatus, lighting module, and lighting method of the present disclosure provide near infrared rays of a single wavelength within a range from 600 nanometers to 1400 nanometers in a continuous or discontinuous mode. When in use, the light of each light-emitting component is directed toward the foramen magnum of a patient to facilitate the penetration of the light into the brain. The lighting of near infrared rays is further combined with the drinking of the hydrogen water or inhaling of the hydrogen gas to improve the physical functions of PD patients. Therefore, the lighting mechanisms (the lighting apparatus, lighting module and lighting method) of the present disclosure can be implemented in a simple way to conduct light-hydrogen therapy, and the lighting mechanisms of the present disclosure allow PD patients to conduct the therapy at home.

The foregoing embodiments are used to illustrate the principles and functions of the present disclosure, rather than to restrict the present disclosure. Those who are familiar with this art can modify the foregoing embodiments without departing from the spirit and scope of the present disclosure. Therefore, the scope of protection of the present disclosure should be accorded with the listed claims described hereinafter.

What is claimed is:

1. A lighting module, comprising:
a light-emitting device providing light in a discontinuous emission mode, wherein the light is near infrared rays or visible red light; and
a container containing a hydrogen-containing fluid to work together with the light-emitting device via an intake of the hydrogen-containing fluid.

2. The lighting module of claim 1, wherein the light-emitting device provides the light of a single wavelength.

3. The lighting module of claim 1, wherein the light-emitting device provides the light of different wavelengths.

4. The lighting module of claim 1, wherein the light-emitting device comprises one or more diodes generating the near infrared rays of a single wavelength within a range from 600 to 1400 nanometers.

5. The lighting module of claim 1, wherein the hydrogen-containing fluid is hydrogen water or hydrogen gas, and wherein the hydrogen water is regular water dissolved with 0.1 ppm to 6 ppm of hydrogen and the hydrogen gas is regular air mixed with 0.1% to 3.8% of hydrogen.

6. The lighting module of claim 1, further comprising a power source electrically connected to the light-emitting device, wherein the power source provides a function of grounding and is electrically connected to an external surface of the container or a ground of the container.

7. The lighting module of claim 6, wherein current from the power source to the electrically connected light-emitting device is supplied intermittently.

8. A lighting apparatus, comprising:
a carrier; and
the lighting module of claim 1 integrated into the carrier.

9. The lighting apparatus of claim 8, wherein the carrier exhibits a chair structure.

10. The lighting apparatus of claim 8, wherein the light-emitting device of the lighting module provides the light of a single wavelength.

11. The lighting apparatus of claim 8, wherein the light-emitting device of the lighting module provides the light of different wavelengths.

12. The lighting apparatus of claim 8, wherein the light-emitting device of the lighting module comprises diodes capable of emitting the visible red light or the near infrared rays of a single wavelength within a range from 600 to 1400 nanometers.

13. The lighting apparatus of claim 8, wherein the hydrogen-containing fluid of the lighting module is hydrogen water or hydrogen gas, and wherein the hydrogen water is regular water dissolved with 0.1 ppm to 6 ppm of hydrogen and the hydrogen gas is regular air mixed with 0.1% to 3.8% of hydrogen.

14. The lighting apparatus of claim 8, further comprising a power source electrically connected to at least one of the light-emitting device of the lighting module and the container of the lighting module.

15. The lighting apparatus of claim 14, wherein current from the power source to the electrically connected light-emitting device is supplied intermittently.

16. A lighting method, comprising:
providing a light source emitting light in a discontinuous mode on a lower back part of a head of a human body and next to a back of a neck of the human body, wherein the light emitted by the light source is near infrared rays or visible red light; and inhaling or drinking a hydrogen-containing fluid.

17. The lighting method of claim 16, wherein the light source provides the light of a single wavelength.

18. The lighting method of claim 16, wherein the light source provides the light of different wavelengths.

19. The lighting method of claim 16, wherein wavelength of the near infrared rays or the visible red light is within a range from 600 to 1400 nanometers.

20. The lighting method of claim 16, wherein the hydrogen-containing fluid is hydrogen water or hydrogen gas, and wherein the hydrogen water is regular water dissolved with 0.1 ppm to 6 ppm of hydrogen and the hydrogen gas is regular air mixed with 0.1% to 3.8% of hydrogen.

21. The lighting method of claim 16, further comprising providing the lighting module of claim 1, wherein the light-emitting device of the lighting module serves as the light source, and the container of the lighting module contains the hydrogen-containing fluid.

22. The lighting method of claim 21, wherein the lighting module further comprises a power source electrically connected to the light-emitting device and the container.

23. The lighting method of claim 22, wherein current from the power source to the electrically connected light-emitting device is supplied intermittently.

24. The lighting method of claim 16, wherein the light source emits the light into a brain through a foramen magnum.

25. The lighting method of claim 16, wherein the light source emits the light into a brain through a foramen magnum and surrounding holes of the foramen magnum.
